(19) United States
(12) Patent Application Publication
(10) Pub. No.: US 2014/0276101 A1
(43) Pub. Date: Sep. 18, 2014

(54) MEDICAL DEVICE FOR DETECTING A TARGET AND RELATED METHODS OF USE

(71) Applicant: Boston Scientific Scimed, Inc., Maple Grove, MN (US)

(72) Inventors: William M. ASSELIN, Lunenburg, MA (US); Carey B. MANNING, Brighton, MA (US)

(73) Assignee: Boston Scientific Scimed, Inc., Maple Grove, MN (US)

(21) Appl. No.: 14/209,182

(22) Filed: Mar. 13, 2014

Related U.S. Application Data
(60) Provisional application No. 61/785,320, filed on Mar. 14, 2013.

(51) Int. Cl. A61B 18/24; A61B 5/107; A61B 8/12; A61B 5/00

(52) U.S. Cl. CPC A61B 18/245; A61B 5/0084; A61B 5/4887; A61B 5/4836; A61B 5/742; A61B 5/7455; A61B 5/7405; A61B 5/1076; A61B 5/1079; A61B 8/12
USPC 600/476; 606/2.5; 600/407

(57) ABSTRACT

A medical device may include an elongate member having a proximal end, a distal end, and a lumen extending therebetween, wherein the elongate member may be configured for insertion into a patient. The medical device may also include a detection member configured to detect at least one of a location, geometry, or distance of a target relative to the distal end of the elongate member, the target being at least one of a stone, a foreign object, or a tissue within a body cavity. The detection member may include a transmitting element and a receiving element.

This application claims the benefit of U.S. Provisional Application No. 61/785,320, filed Mar. 14, 2013, the disclosure of which is incorporated herein by reference in its entirety.

### FIELD

This disclosure relates generally to systems and methods for minimally invasive medical procedures within a patient's body cavity. More particularly, embodiments of the present disclosure relate to devices and methods to identify the location of a target (e.g., a stone, foreign object, and/or tissue) within a patient's body.

### BACKGROUND

Urolithiasis is a condition in which a kidney stone forms within a person's urinary tract. A kidney stone is a small hard stone that can cause pain, bleeding, obstruction, or infection. A kidney stone forms from deposits of calcium, phosphates, and urates. A treatment procedure for urolithiasis is kidney stone ablation using a mechanism such as a laser or lithotripter to ablate and/or fragment the kidney stone, after which the resulting fragments may be removed from the body and/or eliminated along with urine. A flexible fiber-optic endoscopic device delivers energy, such as, e.g., laser, radio frequency, ultrasound, etc., to the kidney stone.

The distance between the energy delivery device and the kidney stone affects the amount of energy effectively transmitted, which in turn affects quality of fragmentation. Generally, each energy source has an optimum range from its target. At this range, the energy source operates to achieve maximum disruption of the target stone. In addition, other factors, such as, geometry of the kidney stone and its precise location within the body cavity may affect effective fragmentation.

Further, if the energy delivery device is advanced too close to the kidney stone, the device may be damaged. During operation, the distal end of the energy delivery device may extend out from the distal end of a suitable introduction sheath, e.g., an endoscope, and if that end collides with the kidney stone, the energy delivery device and/or introduction sheath may be damaged. Such damage may cause the delivered energy to be transmitted at undesirable locations, which may cause inadvertent damage to surrounding tissue. Thus, a physician must accurately detect the distance between the energy delivery device and the kidney stone.

Endoscopic visualizing devices, such as a camera or ultrasound device can help physicians roughly estimate the geometry and location of the kidney stone as well as the distance between the energy deliver device and the kidney stone. Visualizing devices, however, have been shown to fall short of providing the degree of detail and precision required to control the urolithiasis procedure. Such inaccuracy may lead to complications or cause inadvertent damage to nearby tissue.

Therefore, there exists a continuing need for devices that can accurately communicate to an operator the distance between the energy delivery device and the kidney stone while performing ablation and/or fragmentation of the kidney stone.

### SUMMARY

Embodiments of the present disclosure provide a medical device for detecting an object within a patient's body.

In accordance with an aspect of the disclosure, a medical device may include an elongate member having a proximal end, a distal end, and a lumen extending therebetween, wherein the elongate member may be configured for insertion into a patient. The medical device may also include a detection member configured to detect at least one of a location, geometry, or distance of a target relative to the distal end of the elongate member, the target being at least one of a stone, a foreign object, or a tissue within a body cavity. The detection member may include a transmitting element and a receiving element.

Various embodiments of the medical device may include one or more of the following features: the elongate member and the detection member may be independently steerable; the transmitting element may direct energy towards the target, and the receiving element may receive energy reflected from the target; the energy may be one of radar or acoustic energy; the transmitting and receiving element may be included within a single housing; the medical device may further include an energy delivery device; the energy delivery device may be a laser fiber; the detection member may be configured to detect at least one of a location, geometry, or distance of a target relative to the distal end of the elongate member based on energy reflected from the target; the medical device may be configured to communicate to a user the distance of the target relative to the distal end of the elongate member by a signal; and the signal may be one of a visual, tactile, and an audible signal.

In another embodiment, a sensing system may include an elongate member having a proximal end, a distal end, and a lumen extending therebetween. The elongate member may be configured to be inserted into a patient. The sensing system may also include a transmitting element carried within the lumen and configured to transmit energy from the distal end of the elongate member toward the target, and a receiving element carried within the lumen and configured to receive transmitted energy reflected from the target, wherein the target is at least one of a stone, a foreign object, or a tissue within a body cavity.

Various embodiments of the system may include one or more of the following features: the transmitting element may be configured to direct at least one of radar, acoustic, radiofrequency, and optical energy at the target; a control module configured to calculate at least one of a location, geometry, or distance of a target relative to the distal end of the elongate member based on energy reflected from the target; and the system may be configured to communicate to a user the distance of the target relative to the distal end of the elongate member by a signal; the signal may be one of a visual, tactile, and an audible signal.

In a further embodiment, a method for detecting an object within a patient's body. The method may include introducing a medical device into a body cavity. The medical device may include an elongate member having a proximal end, a distal end, and a lumen extending therebetween, wherein the elongate member may be configured to be inserted into a patient. The medical device may also include a detection member configured to detect at least one of a location, geometry, or a distance of a target relative to the distal end of the elongate member, the target being at least one of a stone, a foreign object, or a tissue within a body cavity. The detection member may include a transmitting element and a receiving element. The method may also include transmitting an energy signal from the transmitting element, receiving a reflection of the transmitted energy signal with the receiving element, and processing the received reflection to determine at least one of the location, geometry, or distance of the target relative to the elongate member.

Various embodiments of the method may include one or more of the following features: the energy signal may be one of a radar and an acoustic signal; the medical device may be configured to communicate to a user the distance of the target relative to the distal end of the elongate member by a signal; the signal may be one of a visual, tactile, and an audible signal; and the medical device may further include a laser energy delivery device.

It is to be understood that both the foregoing general description and the following detailed description are exemplary and explanatory only and are not restrictive of the disclosure, as claimed.

### BRIEF DESCRIPTION OF THE DRAWINGS

The accompanying drawings, which are incorporated in and constitute a part of this specification, illustrate exemplary embodiments of the present disclosure and together with the description, serve to explain the principles of the disclosure.

FIG. 1 illustrates an exemplary medical device according to an embodiment of the present disclosure.

FIG. 2 is a sectional view of the distal end of the medical device shown in FIG. 1.

FIG. 3 illustrates an exemplary method of using the medical device of FIG. 1.

### DESCRIPTION OF THE EMBODIMENTS

Reference will now be made in detail to embodiments of the present disclosure, an example of which is illustrated in the accompanying drawings. Wherever possible, the same reference numbers will be used throughout the drawings to refer to the same or like parts. It will be understood that “proximal” and “distal,” as used in this disclosure, refers to positions or directions nearer to or farther from the user, respectively.

### Overview

Embodiments of the present disclosure relate to a medical device used for a minimally invasive surgical procedure, the device having an ability to detect one or more of location, geometry, and/or distance of the target from the medical device. The target may be any abnormality, tissue, or object within a body cavity on which the medical device operates. In some embodiments, the target may be a urinary or biliary stone. In other embodiments, the target may be undesired tissue, such as, e.g., a polyp, a cancerous lesion or cluster, a collection of blood, or blood clot. Still further, the target may be a foreign object disposed within the patient's body. The embodiments of the medical device disclosed here relate to ablation of a kidney stone. The medical device detects one or more of location, geometry, and/or distance of the kidney stone (target) from the medical device and then uses this information in ablating the kidney stone.

Although the embodiments disclosed herein are described in connection with a kidney stone as the target and a laser fiber as the energy delivery device, those of ordinary skill in the art will readily recognize that the principles of the present disclosure may be utilized with any suitable energy delivery device relative any target for treatment or diagnostic purposes. Moreover, the principles of the present disclosure may or may not be used in conjunction with energy delivery devices. Stated differently, the principles disclosed herein may be used solely to identify one or more of a location and/or geometry of a target.

In one embodiment of the present disclosure, the medical device includes an elongate member having a proximal end, a distal end, a lumen extending between the proximal and the distal ends. The elongate member may include one or more internal channels for introducing one or more medical tools within a patient's body. A detection member and an energy delivery device (e.g., ablation) tool may be introduced through the one or more channels. In some embodiments, the detection member and energy delivery device may be integrated into a single device. In other embodiments, they may include separate and distinct devices.

In operation, the detection member ensures that the energy delivery device provides an appropriate amount and intensity of energy by locating the target and detecting the distance between the distal end of energy delivery device and the target. In general, the detection member may be configured as an elongate tubular structure having distally mounted radar receiving and transmitting antennas, coupled to external signal generating and processing apparatus. Alternatively, the system could operate based on sonar or some other detection modality, such as, Infrared, LIDAR, or ultrasound. Initially, the energy delivery device may be in an inactive state and the detection member may be operated to locate the target. Upon detection that a target, such as, e.g., kidney stone, is at a desired distance from the energy delivery device, such as, e.g., a laser fiber, the operator activates the energy delivery device to fragment and subsequently remove target from the body.

In the following sections, embodiments of the present disclosure will be described with reference to a procedure to remove kidney stone from a urinary system. It will be understood that this choice is merely exemplary and that the device may be utilized in any other body cavity, such as gastrointestinal tract, biliary canal, or coronary artery, or any other body cavity.

### Exemplary Embodiments

FIG. 1 illustrates an exemplary medical device 100 according to an embodiment of the present disclosure. The medical device 100 may be used for ablation of a kidney stone. The medical device 100 includes an elongate member 102 having a proximal end 104, a distal end 106, a lumen 108 extending between the proximal, and the distal ends 104,106. The proximal end 104 may connect to a handle 110 while distal end 106 may include one or more openings 116 in communication with the surrounding body cavity. The elongate member 102 may include one or more channels 112a-b for advancing desired medical tools into a patient's body. Although the depicted embodiment illustrates only two channels 112a-b, elongate member 102 may include a greater or lesser number of channels 112a-b. A detection member 118 and an ablation tool 120 may be inserted through the channels 112a-b. In some embodiments, detecting member 118 may be integral with elongate member 102. In embodiments where ablation tool 120 is advanced to a target location with a patient's body via an endoscope or suitable introduction sheath, the endoscope or introduction sheath may be integrally formed with detection member 118. Still further, detection member 118 may be provided on an instrument, such as, e.g., a needle, snare, forceps, scissors, capable of performing one or more functions in addition to the detecting function described herein

Elongate member 102 is an elongate tube with a cross-sectional configuration adapted according to a desired body lumen. In the illustrated embodiment, the elongate member 102 includes a substantially circular cross-sectional configuration, with a similarly configured hollow interior lumen 108, although other shapes may be used. The shape, size, length, and structure of the elongate member 102 may be varied depending upon the particular implementation and intended use. For example, elongate member 102 may be rigid along its entire length, flexible along a portion of its length, or configured for flexure at only certain specified locations. The proximal end 104 or distal end 106 may include geometrical structures, such as, rounded or beveled terminal ends and/or faces, to reduce trauma and irritation to surrounding tissues. In addition, the elongate member 102 or a portion of it may be selectively steerable. Mechanisms such as, motors, hydraulic or any other actuators may be used to selectively steer the elongate member 102.

Elongate member 102 may be formed of any suitable material having sufficient flexibility to traverse body cavities and tracts, such as, fiber or wires that may be woven or braided together using synthetic plastics, fiber, or polymers. Alternatively, elongate member 102 may be rigid or semi-rigid, formed from materials, such as, stainless steel or the like, including shape memory alloys, such as, e.g., Nitinol. In general, elongate member 102 may be made of any suitable material that is compatible with living tissue or a living system. Suitable materials may include nitinol, ePTFE, fabric, and suitable nickel and titanium alloys. Those in the art are well aware of the range of suitable and available materials.

Further, the elongate member 102 may include any suitable coating or covering. For example, the outer surface may include a layer of lubricous material to facilitate insertion through a body lumen or surgical insertion. Further, elongate member 102 may be coated with biocompatible antibacterial and/or anti-inflammatory substances, if desirable.

Elongate member 102 may be provided with any of a wide variety of end-effector devices for performing a range of tasks including fragmenting kidney stones. In an embodiment of the present disclosure, the ablation tool 120 may be configured as a laser fiber disposed within a channel 112b and connected to a laser system (not shown) through ports 124. The laser system directs laser energy through the laser fiber 120 (ablation tool), and this energy may be directed longitudinally or laterally from the distal end 106. Alternatively, the laser fiber 120 may be formed integrally into the elongate member 102. Laser fiber 120 can be sized in an approximate range of about 200 to about 500 microns for the desired purpose. The laser system itself may be, for example, a coumarone-based pulsed dye laser, or a laser formed of alexandrite, Ho:YAG, or Nd:YAG.

To facilitate ablation, embodiments of the present disclosure may employ the detection member 118, located at the distal end 106 of elongate member 102. In general, the detection member 118 directs some form of wave energy, such as radar, sonar, radiofrequency, radiation, and/or optical energy, at a target stone and receives reflected energy from the stone. In some embodiments, the energy may be delivered as mechanical vibration and/or thermal energy, which may be delivered continuously or in a pulsed fashion. A control module 122 includes a computing device that analyzes the reflected energy to determine the location and/or geometry of the target as well as the distance between the target and distal end 106. In one embodiment, for example, the control module 122 may be configured to detect changes in reflected energy profiles. For example, energy (e.g., laser or sound energy) delivered in a pulsed fashion may be reflected a differing or varying rates or altogether randomly when compared to energy delivered continuously. Those of ordinary skill in the art will understand that the reflected energy that is received by device 100 may be in a form that is different than the form of the transmitted energy. For example, a laser energy delivered to ablate a stone may be received as light, heat, and sound energy, each of which may be analyzed to identify one or more characteristics of the stone. In the illustrated embodiment, the system is configured as a radar-based device. The following sections describe the system member in greater detail.

In some embodiments, the medical device 100 may additionally or alternatively include a plurality of temperature sensing elements for detecting thermal energy reflected by a stone, for example. The temperature sensing elements may include thermocouples, thermistors, or any or suitable devices known in the art. Further, medical device 100 may additionally or alternatively include a projecting member (not shown), such as, e.g., a small wire, that extends distally of distal end 106. The projecting member may be configured to engage a stone or other material disposed in a patient's lumen, thereby detecting the presence or absence of stone via, for example, mechanical engagement or vibration.

FIG. 2 represents a longitudinal cross-section of a distal portion of the medical device 100. The detection member 118 may include, but is not limited to, an elongate tube 202, a radar-transmitting antenna 204, and a radar-receiving antenna 206, both disposed at the distal end of the elongate tube 202. Although the depicted embodiment illustrates the radar-transmitting antenna 204 and radar-receiving antenna 206 as separate, discrete structures, those of ordinary skill in the art will recognize that the radar-transmitting antenna 204 and radar-receiving antenna 206 may be provided within a single, multi-functional component. The elongate tube 202 may be slidably disposed within a channel 112a.

The radar signal is generated in the control module 122. The control module 122 may be disposed internally or externally to the medical device 100. In the illustrated embodiment, the control module 122 is disposed externally to the medical device 100 and patient. The control module 122 may generate the radar signal using a radar generation circuitry, which may be any electronic circuit that may generate radar signals of a desired frequency band. The radar generation circuit may include electronic devices such as crystal oscillators, POT (Power Oscillation Transmitters), PAT (Power Amplifier Transmitters), VCO (Voltage-Controlled Oscillator) or a PLL (Phase Lock Loop) and the like. The control module 122 may also include a radar receiver circuit. An appropriate communication medium 208, such as copper wire, coaxial cable, or wireless transmission, may link the antennae 204, 206 to control module 122. The control module 122 and the communication medium 208 will be discussed later in greater detail.

The elongate tube 202 may be the distal portion of the elongate member 102, or it could be a separate tubular device carried within elongate member 102 and deployed in the proximity of the target kidney stone 210. In that scenario, the elongate tube 202 may be a polymeric or rubber fiber or cable covering the components of the detection member 118, to protect the detection member 118 from the fluid medium generally present within a body cavity. Elongate tube 202 may or may not include one or more lumen/channel therethrough. In embodiments where elongate tube 202 includes one or more lumens, a distal end portion of elongate tube 202 may include one or more openings. The elongate tube 202 may include mechanisms such as control lines or wires, or similar actuators, allowing an operator to steer its distal end within the body cavity. This steerability may be independent of the steerability of the elongate member 102, permitting an operator to steer the distal end of the elongate tube 202 around bends or into branching passages separately from the general control of the elongate member 102.

As discussed, the detection member 118 may include the radar transmitting and receiving antennae 204, 206 disposed at the distal end 106 of elongate tube 202. The radar transmitting and receiving antennae 204, 206 may be any type of radar antennae suitable for the desired application of the detection member 118 and the nature of the target to be identified.

The frequency and wavelength of the transmitted and received radar by the transmitting antenna 204 and receiving antenna 206 respectively may depend upon the size and location of the desired target, for example, the kidney stone 210. In general, the diameter of the kidney stone 210 may be greater than the wavelength of the radar transmitted, to detect the size, location, and geometry of the kidney stone 210 with adequate resolution. In a typical renal cavity, the kidney stone 210 may range from a few millimeters (mm) to a few centimeters (cm) in diameter. Thus, radar having a wavelength in the range of a few mm to a few cm may be used to detect the kidney stone 210. As radar may be generally categorized into specific frequency bands, some suitable frequency bands that may be used for detection of kidney stone 210 may be mm (frequency range-40-300 GHz, wavelength range −1 mm-7.5 mm), V (frequency range-40-75 GHz, wavelength range −4 mm-7.5 mm), W (frequency range-75-110 GHz, wavelength range −2.7-4.0 mm), UWB (frequency range-1.6-10.5 GHz, wavelength range −18.75 cm-2.8 cm).

In principle, the detection member 118, which is configured as radar may operate by transmitting, e.g., radio waves using the transmitting antenna 204. The radar may be reflected from the kidney stone 210 and is received by the receiving antenna 206. The control module 122 then analyzes the characteristics of the received radar in connection with the characteristics of transmitted radar to detect size, location, and geometry of the kidney stone 210 using a radar detection algorithm. The control module 122 may use a number of radar detection algorithms known in the art, such as pulse radar (boundary scattering transforms), synthetic aperture radar (SAR), or inverse synthetic aperture radar (ISAR) to detect the kidney stone 210. The radar detection algorithm may include signal processing functions including, but not limited to, removal of noise artifacts, amplification, etc.

As described earlier, the communication medium 208 operationally connects the transmitting antenna 204 and the receiving antenna 206 to the control module 122. The communication medium 208 may be any wired or wireless connection, such as, copper wires, optical fiber, WIFI, Bluetooth or infrared connection. For example, the communication medium 208 may be a length of copper wires operationally connecting the transmitting antenna 204 and the receiving antenna 206 to the control module 122.

In addition, a suitable coating and/or covering may be applied on the detection member 118. For example, the outer surface may include a layer of lubricous material to facilitate its deployment through the channel 112a. Further, the detection member 118 may be coated with a biocompatible material such as Teflon. To inhibit bacterial growth in the body cavity, an outer surface of detection member 118 may be coated with an antibacterial coating.

The control module 122 may implement the desired radar detection algorithm, and control a number of operations, such as, transmission, reception, and processing of the radar signals. In addition, the control module 122 may control other auxiliary devices and functions, such as, an output display or an alarm. Further, the control module 122 may send control signals to actuate other endoscopic medical tools, for example a resection device, an ablation device, or a visualization device.

The control module 122 may include a computing device (not shown) and multiple peripheral tools (not shown). The computing device may be a microcontroller circuit or a microcomputer running custom software. Among other functions, the computing device may control the detection member 118 as well as any signal processing functions. Further controls may extend to other peripheral tools, such as a display device, an audio output device, or an actuator device. The display device may be a CRT monitor, a LCD monitor, or similar device.

In addition, the control module 122 may employ an audio signal to provide direct system performance feedback. It is desirable, for example, to position the distal tip of the elongate tube 202 at an optimal distance from a target stone, and that distance can be indicated by an audio signal. In one embodiment of the disclosure, the control module 122 determines the distance between the distal tip and the target stone, compares that distance to an optimal distance, and indicates the difference value by outputting an audio signal whose pitch, timbre, or rhythm varies with the difference value. The signal may be a series of beeps, for example, whose spacing or pitch varies as the distal tip approaches the optimum position relative to the kidney stone 210. Because operators particularly seek to avoid a collision between the elongate tube 202 and the kidney stone 210, control module 122 may be programmed to sound an alarm when the separation distance goes below a given value.

Further, the control module 122 may control other tools present in other channels 112. For instance, the control module 122 may trigger the laser fiber 120, or other supporting tools required to accomplish a medical procedure. In addition, the control module 122 may include additional equipment, such as, motors or actuators to control other subsidiary operations.

In an alternative embodiment of the present disclosure, the detection member 118 may utilize a sonar transmitter and receiver to detect one or more of the location, geometry, and/or distance of a target from the laser fiber 120. Similarly, another alternative embodiment may employ a LIDAR system as the detection member 118. Further, a person skilled in the art may find many systems and devices that may be used as the detection member 118.

FIG. 3 depicts the medical device 100 within a urinary tract 300, illustrating an exemplary method of using the medical device 100 to fragment and assist in the removal of the kidney stone 210. An operator may insert the elongate member 102 within the urinary tract 300 though a natural orifice or an incision. The operator may then maneuver the elongate member 102 within range of the kidney stone 210, employing the techniques of the present disclosure to prevent the distal end 106 from contacting the stone, while at the same time ensuring that the distal end 106 and/or the distal end of the energy delivery device is disposed at a location optimally spaced from the target.

Returning to FIG. 2, the control module 122 generates a signal and sends it to the transmitting antenna 204 to transmit a radar signal from the distal end 106 through opening 116. The transmitted radar signal impinges on the surrounding cavity and reflects signals back to the receiving antenna 206 through opening 116. Depending upon the wavelength, intensity, and angle of the transmitted and reflected radar signals, the location, geometry, and/or distance of the kidney stone 210 can be determined. Specifically, receiving antenna 206 receives the reflected radar signals and passes it on to the computing device within the control module 122 for processing.

The computing device may then calculate the location, geometry, and/or distance of the kidney stone 210 from the distal end 106 using one or more radar detection algorithms. The computing device may further generate any graphical, numeric, or audio output at any attached output device within the control module 122. The graphical or numeric output generated by the computing device may be displayed on the display device, such as an LCD monitor, to aid the operator in positioning the distal end 106 relative to the kidney stone 210. During that process, as discussed above, an audio signal may vary in pitch, timbre, or rhythm, guiding the operator toward the optimum position. The audio signal may also sound an alarm if the distal end 106 approaches to within a predetermined danger distance from the kidney stone 210, thereby assisting to prevent inadvertent collision with the kidney stone 210.

Referring to FIG. 3, the operator may thus position the distal end 106 at an optimum distance and at an optimum angle from the kidney stone 210 for efficient ablation. The operator may then ablate the kidney stone 210 using the laser fiber 120 (ablation tool). The laser energy may exit the elongate member 102 through opening 116.

After performing the ablation, the detection member 118 may confirm successful ablation of the kidney stone 210, employing the radar signals to determine the size of any remaining of the original kidney stone 210. After completion of the ablation and related procedures, the operator may finally retract the medical device 100 from the urinary tract 300.

It may be noted that the use of the medical device 100 mentioned above is an exemplary description of use and a person skilled in the art may envision other uses for the medical device 100. For instance, the disclosed device may be used in the coronary artery to detect plaque buildup. The detection member 118 may be used to detect location, size, and/or geometry of the plaque buildup within the coronary artery while a medical tool may be inserted within channels 112 to remove the plaque buildup. Other instances may include application of the disclosed device in other body cavities, such as, gastrointestinal tract or the biliary system.

Embodiments of the present disclosure may be used as described herein or in combination with any suitable imaging modality known in the art. For example, the principles of the present disclosure may be used in conjunction with one or more internal and/or external imaging modalities (e.g., ultrasound) to enhance the detection of, e.g., stones with a patient.

Embodiments of the present disclosure may be used in any medical or non-medical procedure, including any medical procedure that requires detection of location, geometry, and/or distance of a target from the medical device 100 within a body cavity. In addition, at least certain aspects of the aforementioned embodiments may be combined with other aspects of the embodiments, or removed, without departing from the scope of the disclosure.

Other embodiments of the present disclosure will be apparent to those skilled in the art from consideration of the specification and practice of the embodiments disclosed herein. It is intended that the specification and the examples described herein be considered as exemplary only, with a true scope and spirit of the disclosure being indicated by the following claims.

What is claimed is:

1. A medical device comprising:
an elongate member having a proximal end, a distal end, and a lumen extending therebetween, wherein the elongate member is configured for insertion into a patient; and
a detection member configured to detect at least one of a location, geometry, or distance of a target relative to the distal end of the elongate member, the target being at least one of a stone, a foreign object, or a tissue within a body cavity, wherein the detection member includes a transmitting element and a receiving element; and
an energy delivery device.

2. The medical device of claim 1, wherein the elongate member and the detection member are independently steerable.

3. The medical device of claim 1, wherein the transmitting element directs energy toward the target, and the receiving element receives energy reflected from the target.

4. The medical device of claim 3, wherein the energy is one of radar or acoustic energy.

5. The medical device of claim 1, wherein the transmitting and receiving elements are included within a single housing.

6. The medical device of claim 3, wherein the received energy is in a form that is different than a form of the transmitted energy.

7. The medical device of claim 1, wherein the energy delivery device is a laser fiber.

8. The medical device of claim 3, wherein the detection member is configured to detect at least one of a location, geometry, or distance of a target relative to the distal end of the elongate member based on energy reflected from the target.

9. The medical device of claim 1, wherein the medical device is configured to communicate to a user the distance of the target relative to the distal end of the elongate member by a signal.

10. The medical device of claim 9, wherein the signal is one of a visual, tactile, and an audible signal.

11. A sensing system, comprising:
an elongate member having a proximal end, a distal end, and a lumen extending therebetween, wherein the elongate member is configured to be inserted into a patient;
a transmitting element carried within the lumen and configured to transmit energy from the distal end of the elongate member toward the target; and
a receiving element carried within the lumen and configured to receive transmitted energy reflected from the target;
wherein the target is at least one of a stone, a foreign object, or a tissue within a body cavity.

12. The system of claim 13, wherein the transmitting element is configured to direct at least one of radar, acoustic, radiofrequency, and optical energy at the target.

13. The system of claim 12, further comprising a control module configured to calculate at least one of a location, geometry, or distance of a target relative to the distal end of the elongate member based on energy reflected from the target.

14. The system of claim 12, wherein the system is configured to communicate to a user the distance of the target relative to the distal end of the elongate member by a signal.

15. The medical device of claim 9, wherein the signal is one of a visual, tactile, and an audible signal.

16. A method for detecting an object within a patient's body, the method comprising:
introducing a medical device into a body cavity, the medical device including
an elongate member having a proximal end, a distal end, and a lumen extending therebetween, wherein the elongate member is configured to be inserted into a patient; and
a detection member configured to detect at least one of a location, geometry, or a distance of a target relative to the distal end of the elongate member, the target being at least one of a stone, a foreign object, or a tissue within a body cavity,
wherein the detection member includes a transmitting element and a receiving element;
transmitting an energy signal from the transmitting element;
receiving a reflection of the transmitted energy signal with the receiving element; and
processing the received reflection to determine at least one of the location, geometry, or distance of the target relative to the elongate member.

17. The method of claim 16, wherein the energy signal is one of a radar and an acoustic signal.

18. The method of claim 16, wherein the medical device is configured to communicate to a user the distance of the target relative to the distal end of the elongate member by a signal.

19. The method of claim 16, wherein the signal is one of a visual, tactile, and an audible signal.

20. The method of claim 16, wherein the medical device further includes a laser energy delivery device.
